(19) United States
(12) Patent Application Publication
(10) Pub. No.: US 2012/0164755 A1
(43) Pub. Date: Jun. 28, 2012

(54) Selective Ion Binding Nanomaterials

(75) Inventors: Eun Seon Cho, Ecublens (CH); Francesco Stellacci, Pully (CH); Pietro Cesti, Trecate (IT); Raffaella Borrelli, San Germano Vercellese (IT)

(73) Assignees: ENI SPA, Rome (IT); Massachusetts Institute of Technology, Cambridge, MA (US)

(21) Appl. No.: 13/338,394

(22) Filed: Dec. 28, 2011

Related U.S. Application Data
(60) Provisional application No. 61/427,541, filed on Dec. 28, 2010.

(51) Int. Cl. G01N 33/20; B82Y 15/00

(52) U.S. Cl. 436/501; 977/773; 977/810; 977/902

(57) ABSTRACT

Nanoparticles with a patterned ligand coat can bind ions selectively. The ligand patterning can arise via self-assembly when two chemically dissimilar (e.g., in size and/or hydrophilicity) ligands are used together. One of the ligands can include one or more moieties capable of interacting with an ion, such as ether oxygens, hydroxyl groups, amine nitrogens, or other groups having a lone pair of electrons. Ion binding can be both selective and reversible.

### CLAIM OF PRIORITY

This application claims priority to U.S. Patent Application No. 61/427,541, filed Dec. 28, 2010, which is incorporated by reference in its entirety.

### TECHNICAL FIELD

The present invention relates to selective ion binding nanomaterials.

### BACKGROUND

Nanoscale materials such as nanoparticles, nanotubes, and nanowires have attracted significant interest due to their unique properties and potential applications in a variety of fields. Due to their size, they show many outstanding properties when compared to bulk materials. Research on nanoparticles such as quantum dots, gold nanoparticles, and magnetic nanoparticles has focused in applications in biomedicine, optics, photovoltaic devices. When such nanoscale materials have heterogeneous structures, they often show distinctive properties. Heterostructured nanowires (e.g., having a heterogeneous composition, such as by doping) display an improved property in FET device with higher carrier mobility. As another example, the electro-optical properties of metallic or semiconductor nanoparticles having a core-shell structure can be tailored by varying the composition or the thickness of the coating.

### SUMMARY

A nanoparticle can be designed to have particular surface properties. In one aspect, a nanoparticle includes a nanoparticle core, a first plurality of ligands on the core, where the first plurality of ligands includes a moiety capable of interacting with an ion, a second plurality of ligands different from the first plurality of the ligands on the core, where the first plurality of ligands and the second plurality of ligands are arranged in a pattern on the core, and where the first plurality of ligands has formula (I):

Z-L-[X—(CR_{d}Rₑ)ᵢ]ⱼ—R₁ (I)

where

Z is —SH, —OH, —NRₐH, —COOH, —P(O)₂OH, —S(O)OH, —S(O)₂OH, —NC, or —CN;

L is a C₁ to C₁₂ alkylene, cycloalkylene, alkenylene, alkynylene, or arylene group, wherein in L is optionally substituted by 0 to 10 groups selected from R_{b};

Each X, independently, is —O—, —S—, or —NRa-;

R₁ is —H, halo, cyano, nitro, —OR_{c}, —SR_{c}, or —NRₐR_{c};

or R₁ is a C₁ to C₆ alkyl, cycloalkyl, alkenyl, alkynyl, aryl, heteroaryl, or heterocyclyl group, wherein each of alkyl, cycloalkyl, alkenyl, alkynyl, aryl, heteroaryl, and heterocyclyl are optionally substituted by 0 to 3 groups selected from R_{b};

each Rₐ, independently, is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};

each R_{b}, independently, is —H, halo, cyano, nitro, —OR_{f}, —SR_{f}, —NR_{f}R_{g}, oxo, alkyl, cycloalkyl, alkenyl, alkynyl, aryl, heteroaryl, or heterocyclyl;

R_{c} is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};

each R_{d}, independently, is —H, halo, or C₁ to C₄ alkyl, wherein alkyl is optionally substituted by halo, —OR_{c}, —SR_{c}, or —NRₐR_{c};

each Rₑ, independently, is —H, halo, or C₁ to C₄ alkyl, wherein alkyl is optionally substituted by halo, —OR_{c}, —SR_{c}, or —NRₐR_{c};

each R_{f}, independently, is —H, alkyl, cycloalkyl, alkenyl, alkynyl, aryl, heteroaryl, or heterocyclyl;

each R_{g}, independently, is —H, alkyl, cycloalkyl, alkenyl, alkynyl, aryl, heteroaryl, or heterocyclyl;

each i, independently, is 0, 1, 2, 3, or 4; and

j is 0, 1, 2, 3, 4, 5, or 6.

The pattern can be a selected arrangement of ligands on a surface of the core.

The arrangement of ligands into a pattern can be a self-assembly process. In other words, when a nanoparticle core is combined with the first and second plurality of ligands, the ligands can become associated with a surface of the core and, without further intervention, produce a pattern. The pattern can be a regular pattern, e.g., one of alternating stripes of the first ligand and the second ligand. The moiety capable of interacting with an ion can include an ether oxygen, a hydroxyl group, or both. The moiety capable of interacting with an ion can include —O—CH₂CH₂—. The second plurality of ligands can be free of a moiety capable of interacting with an ion. The nanoparticle core can be a gold nanoparticle.

The first plurality of ligands can be substantially more hydrophilic than the second plurality of ligands. The second plurality of ligands can have formula (II):

Z-L-[X—(CR_{d}Rₑ)ᵢ]ⱼ—R₁ (II)

where

Z is —SH, —OH, —NRₐH, —COOH, —P(O)₂OH, —S(O)OH, —S(O)₂OH, —NC, or —CN;

L is a C₁ to C₁₂ alkylene, cycloalkylene, alkenylene, alkynylene, or arylene group, to wherein in L is optionally substituted by 0 to 10 groups selected from R_{b};

Each X, independently, is —O—, —S—, or —NRa-;

R₁ is —H, halo, cyano, nitro, —OR_{c}, —SR_{c}, or —NRₐR_{c};

or R₁ is a C₁ to C₆ alkyl, cycloalkyl, alkenyl, alkynyl, aryl, heteroaryl, or heterocyclyl group, wherein each of alkyl, cycloalkyl, alkenyl, alkynyl, aryl, heteroaryl, and heterocyclyl are optionally substituted by 0 to 3 groups selected from R_{b};

each Rₐ, independently, is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};

each R_{b}, independently, is —H, halo, cyano, nitro, —OR_{f}, —SR_{f}, —NR_{f}R_{g}, oxo, alkyl, cycloalkyl, alkenyl, alkynyl, aryl, heteroaryl, or heterocyclyl;

R_{c} is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};

each R_{d}, independently, is —H, halo, or C₁ to C₄ alkyl, wherein alkyl is optionally substituted by halo, —OR_{c}, —SR_{c}, or —NRₐR_{c};

each Rₑ, independently, is —H, halo, or C₁ to C₄ alkyl, wherein alkyl is optionally substituted by halo, —OR_{c}, —SR_{c}, or —NRₐR_{c};

each R_{f}, independently, is —H, alkyl, cycloalkyl, alkenyl, alkynyl, aryl, heteroaryl, or heterocyclyl;

each R_{g}, independently, is —H, alkyl, cycloalkyl, alkenyl, alkynyl, aryl, heteroaryl, or heterocyclyl;

each i, independently, is 0, 1, 2, 3, or 4; and

j is 0, 1, 2, 3, 4, 5, or 6.

The first plurality of ligands having formula (I), and the second plurality of ligands, having formula (II), can have different formulas.

The moiety capable of interacting with an ion can include an ether oxygen, a hydroxyl group, or both. The moiety capable of interacting with an ion can include from 2 to 6 ether, thioether, or amino functionalities. The first plurality of ligands and the second plurality of ligands can have the same values of Z and L. For the second plurality of ligands having formula (II), j can be zero and R¹ can be other than H, halo, unsubstituted alkyl or unsubstituted aryl. The second plurality of ligands can be free of a moiety capable of interacting with an ion. The nanoparticle core can be a gold nanoparticle. Z can be —SH.

The first plurality of ligands can include a thiol group. The second plurality of ligands can include a thiol group. The second plurality of ligands can include a thioalkane.

In another aspect, a method of making a nanoparticle includes contacting a nanoparticle core with a first plurality of ligands, where the first plurality of ligands includes a moiety capable of interacting with an ion, contacting the nanoparticle core with a second plurality of ligands different from the first plurality of the ligands, and where the first plurality of ligands and the second plurality of ligands are selected so as to form a pattern on the core, and where the first plurality of ligands has formula (I) as described above.

The first plurality of ligands can be hydrophilic and the second plurality of ligands can be hydrophobic. The first plurality of ligands can be longer than the second plurality of ligands. The nanoparticle core can be a gold nanoparticle. The second plurality of ligands can have formula (II) as described above. The first plurality of ligands and the second plurality of ligands can have the same values of Z and L. The first plurality of ligands can include a thiol group. The second plurality of ligands can include a thiol group. The second plurality of ligands can include a thioalkane.

In another aspect, a method of selectively binding ions includes contacting an ion-containing composition with a nanoparticle including a nanoparticle core, a first plurality of ligands on the core, where the first plurality of ligands includes a moiety capable of interacting with an ion, a second plurality of ligands different from the first plurality of the ligands on the core, where the first plurality of ligands and the second plurality of ligands are arranged in a pattern on the core, and where the first plurality of ligands has formula (I) as described above.

Selectively binding ions can include binding ions that are chosen to be separated or removed from other ions. For example, ion separation or removal can be useful for purification of materials (i.e., filtration, ion exchange, or remediation of materials), or selective delivery of ions in various pH or temperature environments. Other applications include purification, e.g., of food, chemical materials including chemical precursors for reactions, and pharmaceutical compounds. Purification can include removal of ions or other molecules that are bound to ions through covalent, non-covalent, or ionic bonds or interactions.

The first plurality of ligands and the second plurality of ligands can be selected such that the nanoparticle has a predetermined affinity for a predetermined ion. In another embodiment, the ligands and pattern can be selected to have a predetermined affinity for an ion or a group of ions. The predetermined ion can be Li⁺, Na⁺, K⁺, Rb⁺, Cs⁺, Ca²⁺, Sr²⁺, Cu²⁺, Fe³⁺, Zn²⁺, Cr³⁺, Cd²⁺, or CH₃Hg⁺, or a combination thereof. The method can further include releasing the ions from the nanoparticle after binding. Releasing the ions can include heating the nanoparticle. The second plurality of ligands can have formula (II) as described above. The first plurality of ligands and the second plurality of ligands can have the same values of Z and L. The method can further include binding to the nanoparticle a species associated with the selectively bound ion. The species associated with the selectively bound ion can be a counterion.

The method can further include selectively removing the selectively bound predetermined ions from the ion-containing composition. The method can further include selectively determining the presence, absence, or concentration of the predetermined ions in the ion-containing composition.

Other features, objects, and advantages will be apparent from the description and drawings, and from the claims.

### BRIEF DESCRIPTION OF THE DRAWINGS

FIG. 1 presents the structures of some nanoparticle ligands.

FIG. 2 is a schematic depiction of nanoparticles with different ligand coatings.

FIG. 3A is a TEM image of nanoparticles. FIG. 3B is a graph depicting TGA analysis of nanoparticles.

FIGS. 4A-4C are graphs showing conductivity of nanoparticle solutions in the presence of different ions.

FIGS. 5A-5E are graphs showing conductivity of nanoparticle solutions in the presence of different ions.

FIGS. 6A-6C are graphs showing conductivity of nanoparticle solutions in the presence of different ions.

FIGS. 7A-7C are graphs showing conductivity of nanoparticle solutions in the presence of different ions.

FIG. 8 is a photograph showing formation of precipitate in ion-nanoparticle solutions.

FIG. 9A is a series of photographs showing dissolution of a salt in the presence of a nanoparticle solution. FIG. 9B is a series of photographs showing non-dissolution of a salt in the presence of a nanoparticle solution. FIG. 9C presents spectra of a nanoparticle solution with and without added methylmercury.

FIG. 10 is a graph showing conductivity of nanoparticle solutions in the presence of different ions.

FIGS. 11A-11D are graphs showing conductivity of nanoparticle solutions in the presence of different ions.

FIGS. 12A-12B are graphs showing conductivity of nanoparticle solutions in the presence of different ions.

FIG. 13 is a graph showing conductivity of nanoparticle solutions in the presence of different ions.

FIGS. 14A-14B are graphs showing conductivity of nanoparticle solutions at a series of different temperatures.

### DETAILED DESCRIPTION

In general, a metallic surface can be coated with layer of molecules having an affinity for the metal. In a well known example, gold surfaces can be coated with a layer of thiols; the —SH group of the thiol has an affinity for gold. In particular, gold surfaces can be coated with a monolayer of thiols. Such materials are often referred to as self-assembled monolayers (SAMs).

SAMs can have a homogeneous composition, as in the case of, e.g., a monolayer of hexanethiol on a gold surface. SAMs can also have a homogeneous composition, for example, with a mixture of different compounds bound to a surface. When the compounds are chemically different, they can undergo a phase separation, forming domains in which each compound is primarily in contact with like compounds. On a flat surface, the domains can take on a “worm-like” appearance. See, for example, Mirkin, C. A.; Letsinger, R. L.; Mucic, R. C.; Storhoff, J. J. Nature 1996, 382, 607-609, which is incorporated by reference in its entirety.

On a constrained surface, e.g., the curved surface of a nanoparticle, dissimilar compounds can also undergo a spontaneous phase separation. The curved surface and small size of a nanoparticle can impose additional constraints on the patterning of the phase separation. For example, depending on the size mismatch and immiscibility of the compounds, and also the size of the nanoparticle, distinct patterns, such as “striped” domains may result. See, e.g., Singh, C.; et al. Phys. Rev. Lett. J1—PRL 2007, 99, 226106; Carney, R. P.; et al. J. Am. Chem. Soc. 2007, 130, 798-799; and Jackson, A. M.; Myerson, J. W.; Stellacci, F. Nature Materials 2004, 3, 330 to 336; each of which is incorporated by reference in its entirety.

Thus, for ligands to be “arranged in a pattern on the core” of a nanoparticle, it is not required that the nanoparticle core have a patterned surface, or that the ligands be engineered such that they necessarily form a pattern on any surface. Rather, the pattern can arise spontaneously by self-assembly. A combination of ligands that gives rise to a pattern on a surface of a nanoparticle core need not give rise to the same pattern on another surface—in other words, a combination of ligands that forms a pattern on a nanoparticle core might form a different pattern (including the absence of a pattern) on a different surface, such as a that of a nanoparticle of different size, or a flat (i.e., non-curved) surface.

A striped pattern can be a result of a competition between entropy and enthalpy. When two dissimilar ligands are self-assembled onto nanoparticles, there is a loss in enthalpy when two dissimilar ligands are close, and thus a driving force favoring ligand separation. However, when one ligand is surrounded by dissimilar (e.g., shorter) ligands, there is an increase in free volume for the extra length of the longer ligand, leading to a gain in conformational entropy, over the case when the same ligands are close-packed. The balance between enthalpy loss and entropy gain depends on the characteristics of ligands and the size of nanoparticles. Particularly, in a certain size range of nanoparticles (e.g., in the range of 2 nm to 10 nm, corresponding to a certain curvature range) and with two dissimilar ligands, the increase of entropy plays a more significant role in phase-separation, and the striped domain pattern can be thermodynamically favored. At larger particle sizes (and correspondingly smaller curvatures), the enthalpy loss is dominant. As a result, the worm-like domains can be found with larger particles. Particles smaller than the certain size range can demonstrate bulk phase separation (sometimes referred to as a Janus nanoparticle).

Nanoparticles with a ligand pattern can exhibit interesting, compared to similar unpatterned nanoparticles (i.e., those with only a single type of ligand). For instance, ligand-patterned nanoparticles can show an enhanced cell-permeability, non-monotonic solubility as a function of ligands ratio, and atypical interfacial energy. See, for example, Centrone, A.; et al. Proceedings of the National Academy of Sciences 2008, 105, 9886-9891; Kuna, J. J.; et al. Nature Nanotechnology 2009, 8, 837-842; and Verma, A.; et al. Nature Materials 2008, 7, 588-595; each of which is incorporated by reference in its entirety.

Nanoparticle ligands can generally be described as having two regions: a particle binding region and a functional region. The particle binding region can be simple, e.g., in many cases the particle binding region can be simply a thiol group, —SH. The functional region can have any desired structure that is compatible with the nanoparticles, the other ligands in use, and the chemical environment in which the nanoparticles are to be used. In some cases, the functional region can also be quite simple, for example, a saturated hydrocarbon group, e.g., n-hexyl or n-octyl. In other cases, the functional region can include more complex structure designed to impart a particular property to the nanoparticle. When more than one ligand is to be used with a nanoparticle, the ligands can be selected together to favor the development of ligand patterning on the nanoparticle, e.g., by selecting ligands that differ in size and/or chemical nature. For example, small hydrophobic ligands and large hydrophilic ligands can be combined to promote ligand patterning on a nanoparticle.

As discussed above, the functional region can impart a particular property to the nanoparticle. For example, the functional region can be selected to have electron donating character, so as to favor electrostatic interaction with positive charges (e.g., positively charged ions, such as metal ions). The functional region can include multiple sites of electron donating character, e.g., atoms bearing unshared pairs of electrons, such as ether oxygen atoms, or hydroxyl groups. These functional groups can impart a suite of properties, including hydrophilicity, and positive ion binding. In this way, a ligand can have moiety capable of interacting with an ion.

For example, a functional region including an ether oxygen, a hydroxyl group, or both, can be selected, since ether oxygens and hydroxyl groups have favorable electrostatic interactions with positive charges, and have hydrophilic character (particularly in the context of a hydrocarbon backbone). A repeating oxyalkylene motif (e.g., an oligo- or polyoxyalkylene, such as —[O—(CH₂)₂]ₙ—, —[O—(CH₂)₃]ₙ—, or [O—CH₂CH(CH₃)]ₙ—, can be suitable for this purpose. In each oxyalkylene, n can be 1, 2, 3, 4, 5, or 6. In some embodiments, n can be selected to provide ion-binding selectivity. In other words, in the context of a patterned nanoparticle, certain values of n can favor selective binding of certain ions over others. The selectivity can allow nanoparticles to bind desired ions, even in the presence of other ions of similar charge and size.

When selecting ligands together to favor patterning on a nanoparticle, it can be advantageous to select two ligands which share a structurally similar region but have different functional regions. The ligand can be such that the functional regions are spaced apart from the nanoparticle by the shared structurally similar region. For example, a pair of ligands that promote patterning can share a nanoparticle binding group (e.g., a thiol) and a spacer region, e.g., an alkylene group. In this case, one ligand might have a small hydrophobic functional region, such as, for example, —H, a halogen, a haloalkyl group (which could be a perhaloalkyl group), or a small alkyl group. The other ligand, in contrast, might have a larger hydrophilic functional region. The shared structural regions can allow the dissimilar ligands to interact closely at the nanoparticle surface, but the different functional regions can favor self-segregation, such that a pattern, e.g., of stripes of each type of ligand, arises on the nanoparticle. Once a suitable set of ligands has been selected, the patterning can arise spontaneously, i.e., by self-assembly.

A nanoparticle ligand can have formula (I):

Z-L[X—(CR_{d}Rₑ)ᵢ]ⱼ—R₁ (I)

where

Z is —SH, —OH, —NRₐH, —COON, —P(O)₂OH, —S(O)OH, —S(O)₂OH, —NC, or —CN;

L is a C₁ to C₁₂ alkylene, cycloalkylene, alkenylene, alkynylene, or arylene group, where in L is optionally substituted by 0 to 10 groups selected from R_{b};

each X, independently, is —O—, —S—, or —NRa-;

R₁ is —H, halo, cyano, nitro, —OR_{c}, —SR_{c}, or —NRₐR_{c};

or R₁ is a C₁ to C₆ alkyl, cycloalkyl, alkenyl, alkynyl, aryl, heteroaryl, or heterocyclyl group, where each of alkyl, cycloalkyl, alkenyl, alkynyl, aryl, heteroaryl, and heterocyclyl are optionally substituted by 0 to 3 groups selected from R_{b};

each Rₐ, independently, is —H, alkyl, cycloalkyl, alkenyl, alkynyl, aryl, heteroaryl, or heterocyclyl, where each of alkyl, cycloalkyl, alkenyl, alkynyl, aryl, heteroaryl, and heterocyclyl are optionally substituted by 0 to 3 groups selected from R_{b};

each R_{b}, independently, is —H, halo, cyano, nitro, —OR_{f}, —SR_{f}, —NR_{f}R_{g}, oxo, alkyl, cycloalkyl, alkenyl, alkynyl, aryl, heteroaryl, or heterocyclyl;

R_{c} is —H, alkyl, cycloalkyl, alkenyl, alkynyl, aryl, heteroaryl, or heterocyclyl, where each of alkyl, cycloalkyl, alkenyl, alkynyl, aryl, heteroaryl, and heterocyclyl are optionally substituted by 0 to 3 groups selected from R_{b};

each R_{d}, independently, is —H, halo, or C₁ to C₄ alkyl, where alkyl is optionally substituted by halo, —OR_{c}, —SR_{c}, or —NRₐR_{c};

each Rₑ, independently, is —H, halo, or C₁ to C₄ alkyl, where alkyl is optionally substituted by halo, —OR_{c}, —SR_{c}, or —NRₐR_{c};

each R_{f}, independently, is —H, alkyl, cycloalkyl, alkenyl, alkynyl, aryl, heteroaryl, or heterocyclyl;

each R_{g}, independently, is —H, alkyl, cycloalkyl, alkenyl, alkynyl, aryl, heteroaryl, or heterocyclyl;

each i, independently, is 0, 1, 2, 3, or 4; and

j is 0, 1, 2, 3, 4, 5, or 6.

In some circumstances, Z can be SH. L can be alkylene, for example, C₆ to C₁₂ alkylene, which can include straight chain or branched, substituted or unsubstituted, C₆ to C₈ alkylene. The group —(CR_{d}Rₑ)ᵢ— can, for example, be —(CH₂)₂—, —(CH₂)₃—, or —(CH₂CH(CH₃))—. As explained above, each occurrence of X is independently —O—, —S—, or —NRₐ—, so that a particular ligand may have each X be, for example, —O—, or a particular ligand may have different values of X for different occurrences of X. The total number of carbon atoms in the ligand can be from 4 to 30, from 5 to 25, from 6 to 20, or from 6 to 15.

A nanoparticle ligand can have formula (II):

Z-L-[X—(CR_{d}Rₑ)ᵢ]ⱼ—R₁ (II)

where

Z is —SH, —OH, —NRₐH, —COOH, —P(O)₂OH, —S(O)OH, —S(O)₂OH, —NC, or —CN;

L is a C₁ to C₁₂ alkylene, cycloalkylene, alkenylene, alkynylene, or arylene group, where in L is optionally substituted by 0 to 10 groups selected from R_{b};

each X, independently, is —O—, —S—, or —NRa-;

R₁ is —H, halo, cyano, nitro, —OR_{c}, —SR_{c}, or —NRₐR_{c};

or R₁ is a C₁ to C₆ alkyl, cycloalkyl, alkenyl, alkynyl, aryl, heteroaryl, or heterocyclyl group, where each of alkyl, cycloalkyl, alkenyl, alkynyl, aryl, heteroaryl, and heterocyclyl are optionally substituted by 0 to 3 groups selected from R_{b};

each Rₐ, independently, is —H, alkyl, cycloalkyl, alkenyl, alkynyl, aryl, heteroaryl, or heterocyclyl, where each of alkyl, cycloalkyl, alkenyl, alkynyl, aryl, heteroaryl, and heterocyclyl are optionally substituted by 0 to 3 groups selected from R_{b};

each R_{b}, independently, is —H, halo, cyano, nitro, —OR_{f}, —SR_{f}, —NR_{f}R_{g}, oxo, alkyl, cycloalkyl, alkenyl, alkynyl, aryl, heteroaryl, or heterocyclyl;

R_{c} is —H, alkyl, cycloalkyl, alkenyl, alkynyl, aryl, heteroaryl, or heterocyclyl, where each of alkyl, cycloalkyl, alkenyl, alkynyl, aryl, heteroaryl, and heterocyclyl are optionally substituted by 0 to 3 groups selected from R_{b};

each R_{d}, independently, is —H, halo, or C₁ to C₄ alkyl, where alkyl is optionally substituted by halo, —OR_{c}, —SR_{c}, or —NRₐR_{c};

each Rₑ, independently, is —H, halo, or C₁ to C₄ alkyl, where alkyl is optionally substituted by halo, —OR_{c}, —SR_{c}, or —NRₐR_{c};

each R_{f}, independently, is —H, alkyl, cycloalkyl, alkenyl, alkynyl, aryl, heteroaryl, or heterocyclyl;

each R_{g}, independently, is —H, alkyl, cycloalkyl, alkenyl, alkynyl, aryl, heteroaryl, or heterocyclyl;

each i, independently, is 0, 1, 2, 3, or 4; and

j is 0, 1, 2, 3, 4, 5, or 6.

Generally, the ligand of formula (II) will be different (i.e., have different values of Z, L, X, etc.) than the ligand of formula (I).

In some embodiments, a nanoparticle includes a ligand coat which includes some ligands of formula (I) and some of formula (II), in other words, the nanoparticle has a heterogeneous ligand coat. In such a case, none, some or all of the types of ligands can be described by formula (I). By way of example, a nanoparticle ligand coat can include EG2, which is the compound of formula (I) in which Z is —SH, L is C₆ alkylene (without substitution), each occurrence of X is —O—, each R_{d} and each Rₑ are —H, each i is 2, j is 2, and R₁ is —OR_{c} where R_{c} is —H. A nanoparticle ligand coat can also include hexanethiol, which is a compound of formula (II) in which Z is —SH, L is C₆ alkylene (without substitution), j is 0, and R₁ is —H. A

In some cases, when a nanoparticle ligand coat includes two types of ligands, one of which can be described by formula (I) and the other by formula (II), both types of ligands can have the same values of Z and L. This is the case with (for example) hexanethiol and EG2. In both of these ligands, Z is —SH and L is C₆ alkylene (without substitution). The nanoparticle ligand coat can include more than two types of ligands.

When the ligand binds to the nanoparticle, the particle binding region can undergo a chemical change. For example, when a thiol-containing ligand binds to a gold nanoparticle, the pKₐ of the thiol can change, altering the thiol/thiolate equilibrium. Thus in some cases, a ligand which, in the form of a pure compound, has the formula:

HS-L-[X—(CR_{d}Rₑ)ᵢ]ⱼ—R₁

can, when interacting with (e.g., bound to) the surface of a nanoparticle, can more accurately be described as having the formula:

⁻S-L-[X—(CR_{d}Rₑ)ᵢ]ⱼ—R₁.

When describing a nanoparticle having a ligand coat, all such protonation states of the ligand(s) are contemplated and considered to be within the meaning of formula (I).

The ligands of formula (I) can include polar groups that can interact with ions in solution, e.g., —O—, —S—, or —NRₐ—. In some cases, the ligands of formula (I) can include more than one such polar group. These groups can selected so as to provide an ion-binding site when part of a patterned nanoparticle. For example, a ligand of formula (I) can include one or more ether, thioether, or amino functionalities which have electron-donating character for interaction with positively charged ions. When there are more than one such functionalities, they can be spaced by substituted or unsubstituted alkylene groups, e.g., —CH₂CH₂—.

The ligands of formula (I) and formula (II) can be selected so as to disfavor close interaction between the ligands of formula (I) and formula (II), in particular when the ligands are bound to a surface (such as a nanoparticle surface). In other words, the ligands of formula (I) and formula (II) can be selected to favor the self-interaction of ligands (I) with other ligands (I), and the self-interaction of ligands (II) with other ligands (II). Thus the mingling of ligands (I) and (II) when bound to a surface is energetically disfavored. This effect can be achieved, for example, by providing ligands of formula (I) which have substantially more polar and/or hydrophilic character than the ligands of formula (II).

In some cases, the ligands can be chosen such that one moiety of the ligands of formula (I) and formula (II) allow a degree of close interaction, but other moieties disfavor close interaction sufficiently that the ligands of formula (I) and formula (II) will self-assemble on a surface to preferentially self-interact. For example, in both formula (I) and (II), L can be a hydrophilic moiety (such as C₁ to C₁₂ alkylene, particularly C₄ to C₁₂ alkylene), allowing a degree of close interaction, but formula (I) and (II) can differ in their —[X—(CR_{d}Rₑ)ᵢ]ⱼ—R₁ moieties so as to disfavor close interaction.

In this regard, ligands of formula (I) can have values of L, X, R_{d}, Rₑ, j, j, and R₁ that provide substantially more polar and/or hydrophilic character than the values of L, X, R_{d}, Rₑ, i, j, and R₁ in the ligands of formula (II). In particular the values of L, X, and j can be important. A ligand of formula (I) can have a non-zero value of j, thus requiring the presence of at least one group X which can be —O—, —S—, or —NRₐ— thereby providing a degree of polar and/or hydrophilic character. A ligand of formula (II) can have zero as the value of j, such that no group X is present.

Thus, for example, ligands of formula (I) and (II) can have the same values of Z and L, e.g., where Z is —SH and L is a C₁ to C₁₂ alkylene group; in the ligand of formula (I), j is 1, 2, 3, 4, 5, or 6; but in the ligand of formula (II), j is 0. Furthermore, in the ligand of formula (II), R¹ can be substantially non-polar and/or non-hydrophilic, e.g., R¹ can be H. In this case, the moiety -L-[X—(CR_{d}Rₑ)ᵢ]ⱼ—R₁ in the ligand of formula (I) can be substantially more polar and/or more hydrophilic than the ligand of formula (II). Furthermore, as discussed below, the values of L, X, R_{d}, Rₑ, i, j, and R₁ in the ligand of formula (I) can be selected in a manner that facilitates selective interaction with ions, e.g., selective binding of a predetermined ion.

In some cases, the pattern of ligands on a nanoparticle surface can give rise to properties that are not found in the ligands individually, in non-patterned nanoparticles (e.g., nanoparticles bearing only a single type of ligand, also called a homo-ligand nanoparticle), or in surfaces coated with the combination of ligands, but where a different pattern (or no pattern) arises. For example, certain ligands, when in a stripe structure, can mimic crown ethers. Crown ethers are a group of compounds well-known for selectively chelating metal ions. See, for example, Christensen, J. J.; Hill, J. O.; Izatt, R. M. Science 1971, 174, 459-467; Pedersen, C. J. J. Am. Chem. Soc. 1967, 89, 2495-2496; and Michaux, G.; Reisse, J. J. Am. Chem. Soc. 1982, 104, 6895-6899, each of which is incorporated by reference in its entirety. Related compounds include cyclic polyamines and polythioethers. These can have varying ring sizes, depending on the number of ether (or amine or thio) groups in the macrocycle, and can take on a cage-like form, a group of compounds called cryptands. The key factors to forming a complex with metal ions are the electrostatic force and the size match between the ring and metal ion. For example, 18-crown-6 ether ring can exclusively make a complex with potassium ions rather than other ions.

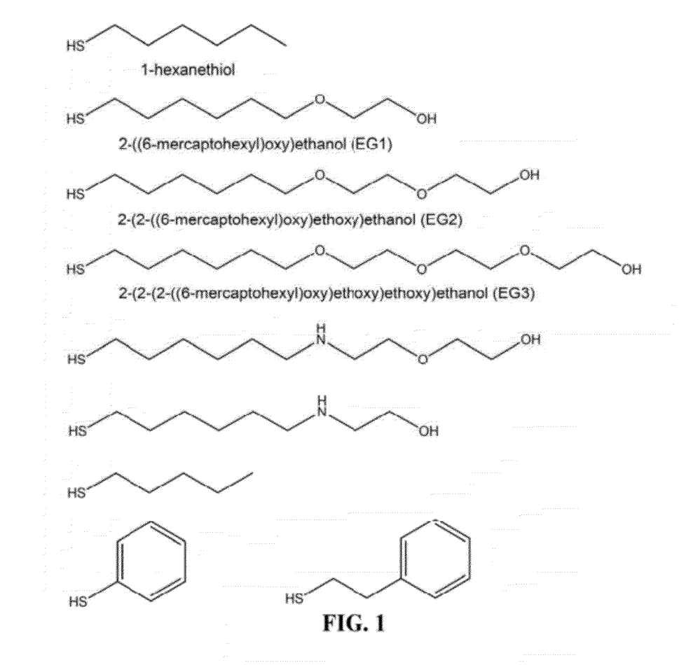

To provide a crown ether-like structure in a striped nanoparticle, alkanethiols can be extended by ethylene glycol groups. For example, the compounds (2-((6-mercaptohexyl)oxy)ethanol (EG1), 2-(2-((6-mercaptohexyl)oxy)ethoxy)ethanol (EG2), and 2-(2-(2-((6-mercaptohexyl)oxy)ethoxy)ethoxy)ethanol) (EG3) can be used as nanoparticle ligands in combination with hexanethiol. The structures of these compounds are shown in FIG. 1. Thus a nanoparticle can be made which has a combination of EG1 and hexanethiol ligands, or a combination of EG2 and hexanethiol, or a combination of EG3 and hexanethiol. In each case, a striped ligand pattern will tend to form because hexanethiol is smaller and more hydrophobic than EG1, EG2, or EG3. The striped pattern can give the nanoparticle different properties than a nanoparticle bearing either ligand alone. The striped nanoparticles also have different properties than a mixture of, for example, hexanethiol homoligand nanoparticles and EG1 homoligand nanoparticles.

By way of example, consider a hexanethiol homoligand nanoparticle. This nanoparticle lacks any moieties capable of any substantial interaction with ions. An EG1 homoligand nanoparticle, in contrast, is replete with ether oxygen atoms and hydroxyl groups; however, (as described below) may show very little or no affinity for ions, possibly because the dense coverage of the nanoparticle with EG1 ligands does not allow enough space for ions to form numerous energetically favorable interactions with the oxygen atoms. But in a striped nanoparticle, the shorter hexanethiol ligands can enforce a degree of spacing between adjacent EG1 ligands. As a result, the striped EG1/hexanethiol nanoparticle can have an affinity for ions that is missing from EG1 alone and is missing from EG1 homoligand nanoparticles.

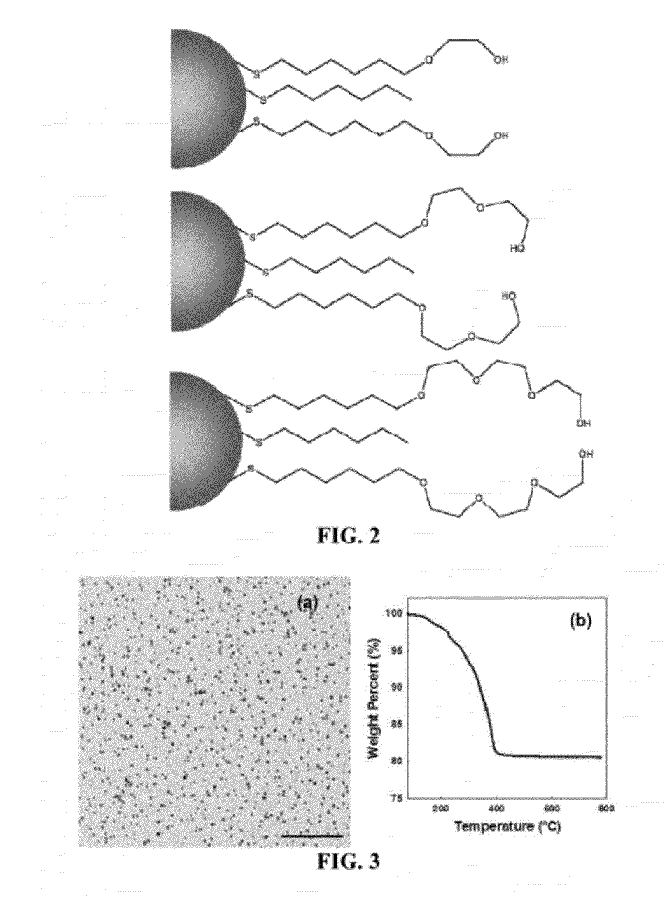

The striped pattern can thus position the oxygen atoms of the hydrophilic ligands in a manner that mimics the positions of oxygen atoms in crown ethers. See FIG. 2, showing how, when in a striped pattern with hexanethiol, EG1 can mimic a 4-oxygen crown ether; EG2, a 6-oxygen crown ether, and EG3, an 8-oxygen crown ether. A set of ligands can be selected such that a patterned nanoparticle has a predetermined affinity for a desired ion or group of ions. The ion can be any ionic species described in the CRC Handbook of Chemistry and Physics, 91st edition (2010), which is incorporated by reference in its entirety. For example, the ion can be Li⁺, Na⁺, K⁺, Rb⁺, Cs⁺, Ca²⁺, Sr²⁺, Cu²⁺, Fe³⁺, Zn²⁺, Cr³⁺, Cd²⁺, or CH₃Hg⁺. In some cases, the nanoparticle can have an affinity for a group of ions, i.e. more than one different ion. A nanoparticle that has an affinity for a particular ion can, in some circumstances, also have an affinity for other ions of similar charge and size, within limits. Thus as described below, one exemplary nanoparticle has an affinity for both Li⁺ and Na⁺, but not for K⁺.

The nanoparticles can be used for the selective removal of ions from a composition. The composition can be any material from which selective ion removal is desired, and includes without limitation aqueous solutions, environmental samples (e.g., soils, natural waters, waste streams), chemicals, foods, and pharmaceuticals. In general, the ion-containing materials is brought into contact with nanoparticles having an affinity for the desired ion. Thus, to remove, for example, methylmercury from an environmental sample, the environmental sample would be brought into contact with nanoparticles having an affinity for methylmercury. Upon contacting, the nanoparticles will bind the desired ions. In some cases, an incubation period, mixing step, or both, can be included. The sample is then separated from the nanoparticles. Separation can include decanting, centrifugation, filtration, or other mechanical separation steps.

In some cases the nanoparticles can be immobilized, e.g., on a support. In this case, separation can be nearly trivial, particularly when the ion-containing composition is a liquid. This situation can resemble filtration or chromatography. Indeed, the nanoparticles can be suitable for use in ion-exchange applications such as ion-exchange chromatography.

The nanoparticles can be used for selectively determining ions in a composition, e.g., determining the presence, absence, or concentration of a predetermined ion in a composition. In one example of selectively determining ions, the nanoparticles can be used to selectively remove ions from a composition as described above, and the nanoparticles recovered in a sample solution. The ions can then be released from the nanoparticles and the concentration of ions determined by any suitable technique. In another example, the nanoparticles can be used to selectively remove ions from a composition as described above, and determined based on changes in the properties of the nanoparticles themselves that occur upon ion binding, e.g., optical or electrical properties.

An ion-binding nanoparticle can also bind other species by virtue of their interaction with the bound ion. In one example, a nanoparticle which is electrostatically neutral can selectively bind positive ions. Maintaining overall charge neutrality, the nanoparticle can also bind negatively charged counterions. This counterion binding can be less specific or non-specific than the primary ion binding, because it relies only on the charge interaction, and not on size and shape considerations, as the primary ion binding does.

### EXAMPLES

### Nanoparticle Synthesis and Characterization

A variety of methods have been developed to synthesize AuNPs. For this work, AuNPs have been synthesized by either a one-phase method in ethanol (see, e.g., Kang, S. Y.; Kim, K. Langmuir 1998, 14, 226-230, which is incorporated by reference in its entirety) or a modified Stucky method (see, e.g., Zheng, N.; Fan, J.; Stucky, G. D. Journal of the American Chemical Society 2006, 128, 6550-6551, which is incorporated by reference in its entirety) in a co-solvent of chloroform/DMF. In the one-phase method in ethanol, 0.45 mmol of HAuCl₄.3H₂O was dissolved in 40 mL of ethanol, followed by the addition of 0.45 mL of thiol mixtures. After 20 minutes of stirring, 5 mmol of NaBH₄ in 75 mL of ethanol was slowly added drop-wise over 50 minutes, and then kept stirring for 3 hours. After completing the reaction, the product was centrifuged and washed with ethanol. In the modified Stucky method, 0.45 mmol of AuPPh₃Cl was mixed with 0.45 mmol of thiol mixture in 40 mL of chloroform/DMF co-solvent, and 5 mmol of tert-butylamine-borane complex was added. The reactant was heated to 55° C. and stirred for 1 hour. After cooling to room temperature, 200 mL of chloroform was added to precipitate out AuNPs, and the product was centrifuged and washed with chloroform and ethanol.

The core size of AuNPs was investigated by JEOL 200cx TEM, and measured by Image J software. The average of the diameter of AuNPs was generally 4 nm (FIG. 3A). In addition, the ligand ratio of the striped AuNPs was confirmed by NMR measurement, after etching the gold core with iodine, which was well-consistent with the synthetic ratio. The organic amount in AuNPs was approximately 20 wt %, as determined by TGA (FIG. 3B).

Ion Binding: Conductivity Tests

A conductivity test was used to examine the interaction of striped and unpatterned gold nanoparticles with different kinds of metal ions. A 0.05 M metal ion solution was added step-wise to a AuNP aqueous solution, with chloride ions or bromide ions as counter-ions.

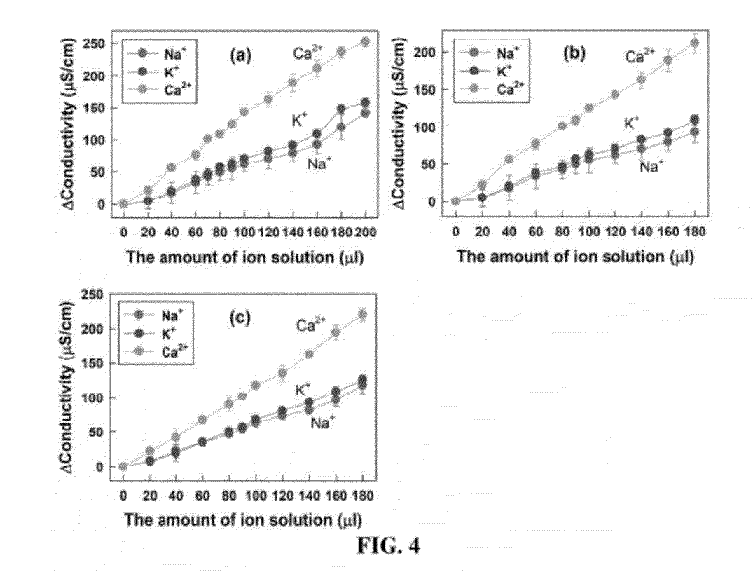

FIGS. 4A-4C show the results for AuNP with a homogeneous (i.e., unpatterned) ligand coat of EG1 (FIG. 4A), EG2 (FIG. 4B), and EG3 (FIG. 4C), for Na⁺, K⁺, and Ca²⁺. In each case, conductivity of the solution increased in approximately linear fashion. Ca²⁺ showed a greater increase in conductivity, as expected to its greater ionic strength.

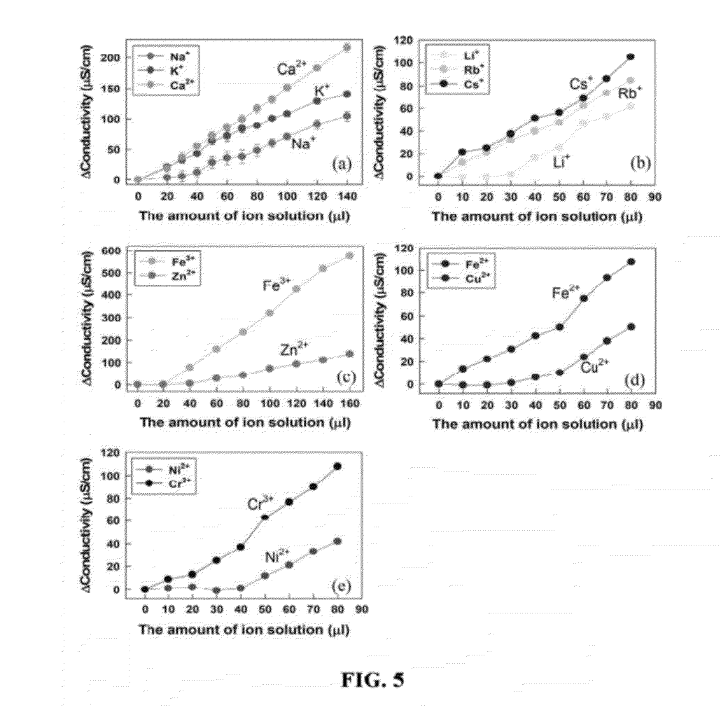

When the test was performed using striped nanoparticles, a different result was observed. FIGS. 5A-5E show results using nanoparticles bearing a striped hexanethiol/EG1 ligand coat. Some plateau regions—indicative of ion binding by the nanoparticles—were observed for sodium, lithium, zinc, iron(III), copper(II) and nickel ions. In contrast, the conductivity increase was insensitive to the presence of the striped nanoparticles for potassium, calcium, rubidium, cesium, iron(II), and chromium ions. It is notable that, for Na⁺, the pattern of conductivity increases differed for striped and unpatterned nanoparticles, which suggested that Na⁺ was bound by the striped nanoparticles. This result indicated that there was no sodium ion-capturing effect for the AuNPs, and that the spacing group (hexanethiol) was important for ion binding.

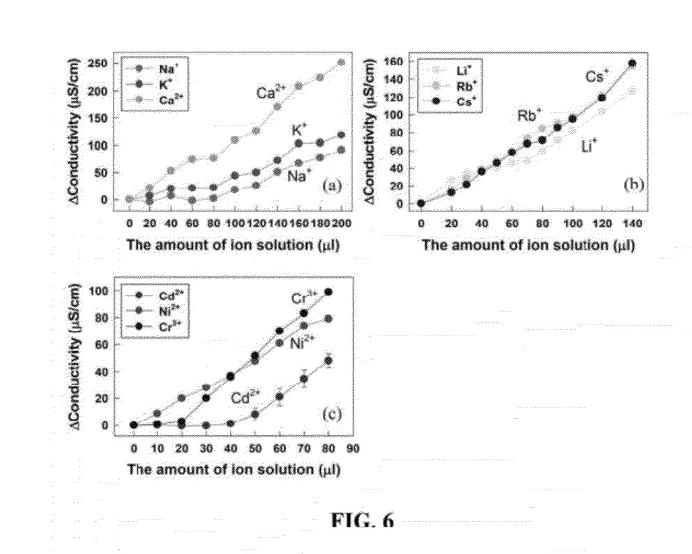

The same test was conducted also for striped nanoparticles having a striped EG2/hexanethiol ligand coat. See FIGS. 6A-6C. Sodium, potassium, chromium, and cadmium ions interacted with these nanoparticles them, but calcium, lithium, rubidium, cesium, and nickel ions did not. As shown in FIG. 4B, for homogeneously EG2 coated AuNPs, there was no ion-capturing phenomenon. In contrast, sodium and potassium ions were captured by striped EG2 AuNPs. This result was consistent with the result for unpatterned and striped EG1 AuNPs. Therefore, it was clear that the striped AuNPs made a complex with a certain kind of metal ions, but corresponding unpatterned AuNPs did not.

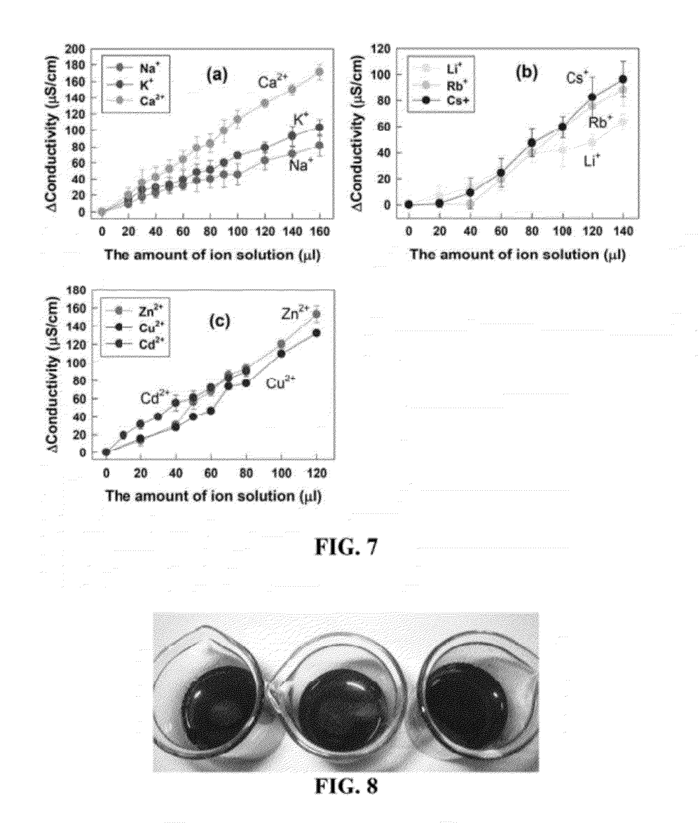

Conductivity was measured in the same way for striped nanoparticles having a striped EG2/hexanethiol ligand coat. See FIGS. 7A-7C. Of the ions tested, only rubidium and cesium ions were captured by the striped nanoparticles, as revealed by the plateau region on the plot.

For each AuNP and ion pair, some precipitation was observed upon ions addition to the AuNP solutions (see FIG. 8). For example, a certain precipitate was formed on the top layer of the solution for striped EG2 AuNPs with sodium (FIG. 8, left) and potassium (FIG. 8, center) ions, but not with calcium (FIG. 8, right) ions. Even though the exact reason for the formation of this precipitate was not determined, it might be related to the ion-capturing effect, and the precipitate would be an indication of the formation of the complex between AuNPs and metal ion, since no precipitation was observed for non-interacting pairs of metal ions and AuNPs.

The results of the conductivity tests for striped nanoparticles are summarized in Table 1.

TABLE 1

| | EG1 | EG2 | EG3 |
|---|---|---|---|
| Li | + | − | − |
| Na | + | + | − |
| K | − | + | − |
| Rb | − | − | + |
| Cs | − | − | + |
| Ca | − | − | − |
| Cu(II) | + | | − |
| Fe(II) | − | | |
| Fe(III) | + | | |
| Zn | + | | − |
| Cr(III) | − | + | |
| Ni | | − | |
| Cd | | + | − |

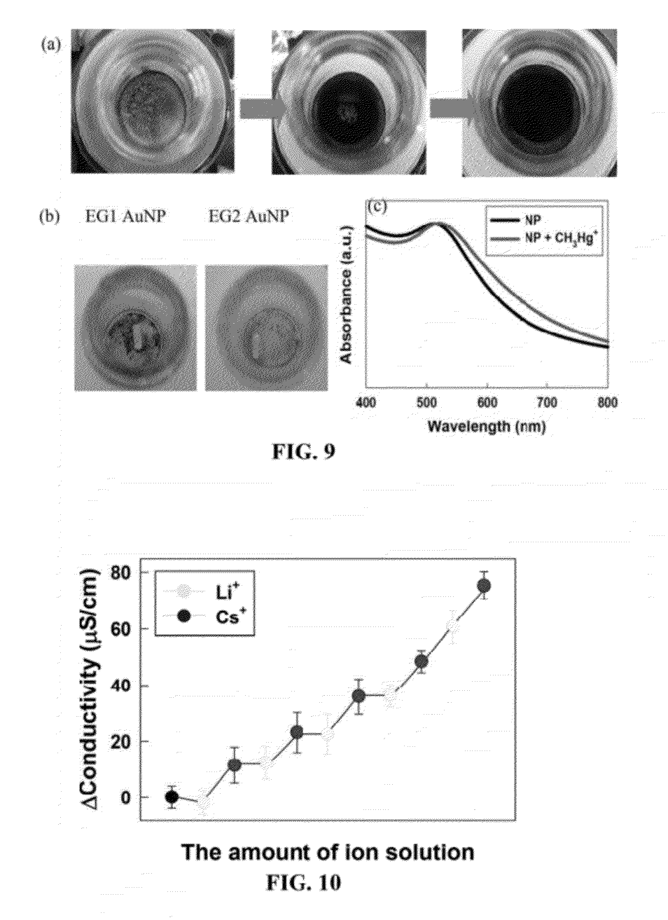

Some salts are only slightly water soluble or even water insoluble. Such materials can be more difficult to remove from the environment compared to more fully water soluble forms salts. For example, salts of methylmercury (CH₃Hg⁺) have very low water solubility. Methylmercury is a typical form of mercury in the food chain, and also one of the most toxic forms of mercury. To determine whether any striped EG AuNPs could interact with methylmercury salts, white methylmercury chloride salts were added to aqueous solutions of striped EG1, EG2, or EG3 AuNPs (FIG. 9). Initially, the white salt was floating on the top layer of solutions, but it disappeared in striped EG3 AuNP solutions after stirring for a time, resulting in them clear solutions (see FIG. 9A). For striped EG1 AuNP and striped EG2 AuNP solutions, however, the white salt remained (FIG. 9B). FIG. 9C shows UV-VIS spectra of the striped EG3 AuNPs, which showed that the plasmon resonance peak for these particles was red-shifted on the addition of methylmercury salt, implying that EG3 AuNPs made a complex with methylmercury ions.

Ion Binding: XPS Analysis

XPS analysis was used to measure the ratio of EG ligand to metal ion. The results for a zinc:striped EG1 AuNP ion pair are shown in Table 2. The results for a methylmercury:striped EG3 AuNP ion pair are shown in Table 3. For the EG1 AuNP and zinc ion complex, the precipitate was obtained from the top layer, which was assumed as a result of AuNP-ion complex, and washed with water to remove free ions. Then, the remaining solid was analyzed with XPS. For the EG3 AuNP and methylmercury ion complex, the uncaptured free methylmercury salt was removed by a 0.2 μm pore size syringe filter, and the filtered solution part was analyzed. The actual sulfur amount was re-calculated based on the amount of other elements, because less sulfur was detected than expected. The decreased detection of sulfur was caused by the proximity of the sulfur atoms to the gold surface of the nanoparticles. The ratio of EG1 ligands to zinc ions was 3.4 and that of EG3 ligands to methylmercury ions was 2.02, using the adjusted sulfur quantity. This suggested that the complex of striped EG1 AuNP and zinc ion would be close to the shape of a cryptand, which is a 3-D form, and that of a striped EG3 AuNP and methylmercury ion would be close to the shape of ordinary crown ether, which is a 2-D form.

TABLE 2

| Conc. at. (%) | C 1s | O 1s | S 2p | Au 4f | Na 1s | Cl 2p | Zn |
|---|---|---|---|---|---|---|---|
| EG1 AuNP + Zn²⁺ | 58.55 | 19.7 | 6.36 | 14.9 | 0 | 0 | 1.37 |

TABLE 3

| Conc. at. (%) | C 1s | O 1s | S 2p | Au 4f | Na 1s | Cl 2p | Hg |
|---|---|---|---|---|---|---|---|
| EG3 AuNP + CH₃Hg⁺ | 30.4 | 60.1 | 0.645 | 2.0 | 0.9 | 6.1 | 0.24 |

Ion Binding Selectivity: Conductivity Tests

To demonstrate that striped nanoparticles can selectively absorb specific ions, conductivity test were carried out with mixture of ions. Based on the conductivity results discussed above, striped EG1 AuNP can bind lithium ions, but not cesium ions.

FIG. 10 shows the measured conductivity of a solution of striped EG1 AuNP to which 10 μl aliquots of lithium and cesium solutions were added alternately. The conductivity did not change when lithium was added, consistent with the lithium ions being bound by the nanoparticles. When cesium was added, however, an increase in conductivity was measured. This pattern continued until total 40 μl of lithium ion solutions were added, corresponding to the stoichiometric point of EG1 AuNP and lithium ion pair, and this point was consistent with the result of FIG. 5B. With this result, it was shown that striped EG AuNPs can bind a certain ion selectively even in the presence of other ions.

Ion Binding: Effect of Spacer Ligand

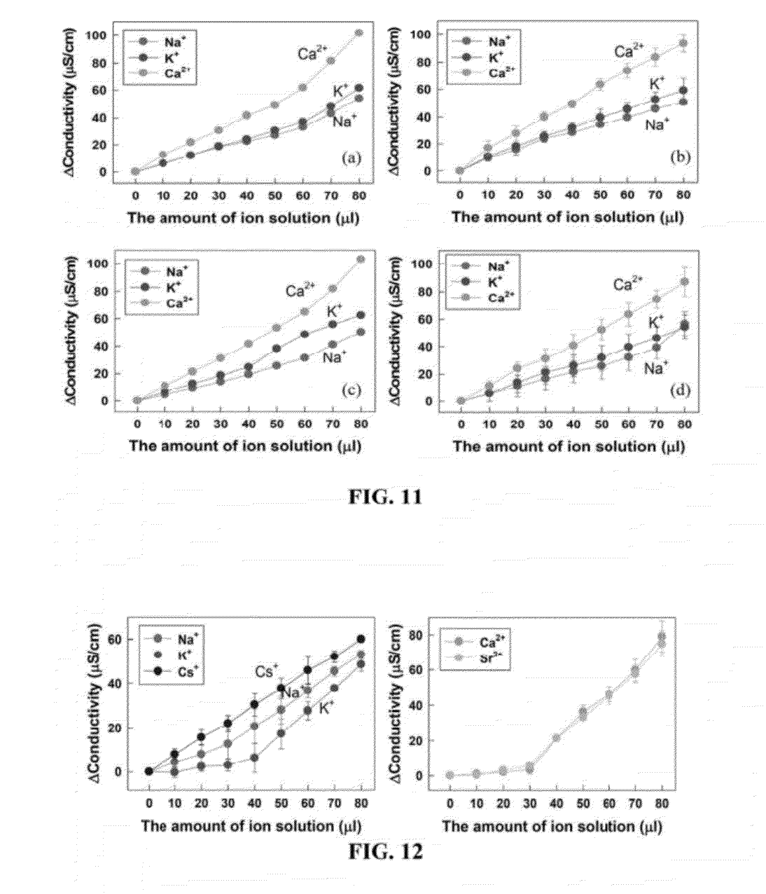

AuNPs with spacing ligands other than hexanethiol were also synthesized, and the conductivity measurement was performed. Two broad types of ligands were used: one with a hydrophilic head such as 6-mercapto-1-hexanol (MHO) or 6-amino-1-hexanethiol (AH), and the other with the aliphatic hydrocarbon chains such as octanethiol (OT) and butanethiol (BT). The experiments with first group were to investigate the influence of the ligand property; the second group was to investigate the effect of spacer ligand length. AuNPs bearing a mixed ligand coat of EG2 and one of the spacer ligands were synthesized and the conductivity tests were done. The results are shown in FIGS. 11A-11D (FIG. 11A, MHO; FIG. 11B, AH; FIG. 11C, OT; FIG. 11D, BT). Remarkably, no evidence of ion binding was observed for any of the four AuNPs, despite striped EG2-hexanethiol AuNPs being able to bind sodium and potassium ions. This indicated that the spacing group plays an important role in ion binding.

More interestingly, striped nanoparticles with OT and EG3 ligands, showed a different effect (FIGS. 12A-12B). AuNPs with OT and EG3 ligands did not capture cesium ions (unlike AuNPs with hexanethiol and EG3), but they were able to bind with potassium, calcium and strontium ions. It is noteworthy that none of the hexanethiol-EG AuNPs were able to capture calcium ions. Thus it is possible that a more diverse set of ions could be selectively captured by altering the ligand group in the striped AuNPs.

In addition, a further experiment was conducted using 3,4-dimethylhexane-1-thiol (brHT), which is a branched form of hexanethiol. It was reported that the branched ligand disturbed the ordered structure on the ligand shell, resulting in the disordered form (see, e.g., Verma, A.; et al. Nature Materials 2008, 7, 588-595, which is incorporated by reference in its entirety). Because ion binding is believed to depend on the ordered patterning of ligands on the nanoparticle surface, it would be expected that using brHT in place of hexanethiol would diminish or destroy the ion binding capability of the nanoparticle.

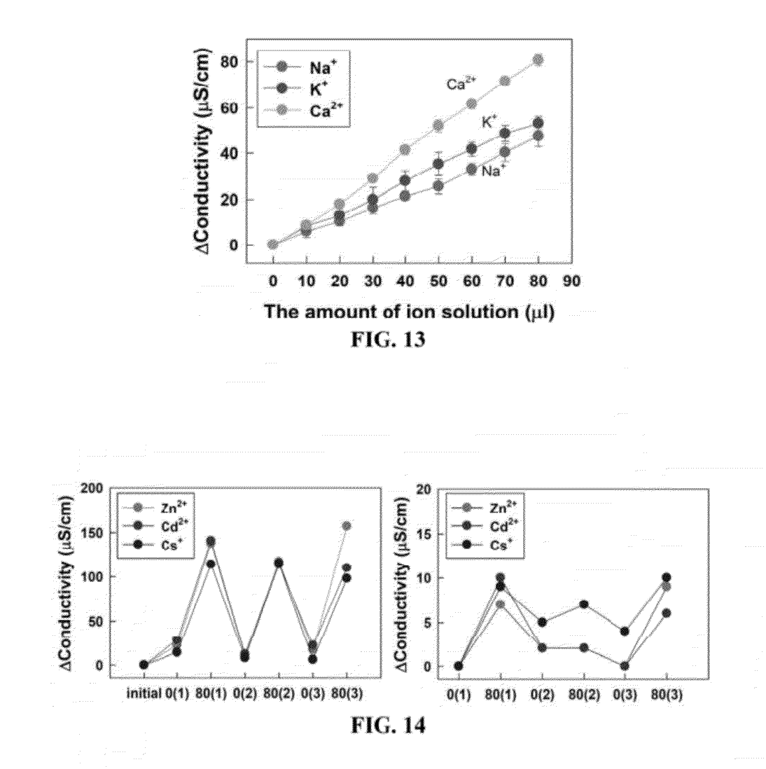

3,4-dimethylhexane-1-thiol (brHT) was synthesized by following the literature and confirmed by NMR spectrum. AuNP with EG2 and brHT ligands was synthesized in the same way that EG2/hexanethiol AuNP was made, and the ratio of two ligands and the size of AuNP were similar. The result of the conductivity test for AuNP with EG2 and brHT was shown in FIG. 13, and there was no capturing effect for this disordered AuNP. This was therefore another indication that the striped structure contributed to the selective ion binding.

Ion Binding: Binding Strength

Enthalpy of K⁺ binding to a variety of materials was investigated by isothermal titration calorimetry. The results are presented in Table 4.

TABLE 4

| material | ΔH (kJ/mol) |
|---|---|
| 18-crown-6 | −21.06 ± 1.11 |
| cryptand | −86.34 ± 12.19 |
| hexanethiol/EG2 AuNP | −63.01 ± 18.39 |
| EG2-only AuNP | −13.42 ± 1.54 |
| free EG2 ligand (with thiol) | −38.17 ± 0.71 |
| free EG2 ligand (without thiol) | −6.77 ± 0.64 |

The potassium ion binds to the EG2 striped AuNPs more strongly than to 18-crown-6, but slightly less than to the cryptand. This suggested that the complex between the EG2 ligand in AuNPs and the potassium ion has a 3-D structure, more closely resembling the cryptand rather than 18-crown-6. Moreover, the EG2 homo-ligand AuNPs showed a very weak binding, which indicated that the striped structure was important for ion-capturing. This result further suggested that having a second, different ligand (e.g., hexanethiol) was also important, as its shorter length facilitated creation of binding pockets. Interestingly, there was a noticeable enthalpy change when adding potassium ions to free EG2 ligand. However, it turned out that this enthalpy change was caused by disulfide bond formation, (i.e., oxidation of free —SH groups), not by interaction with potassium ions.

Ion Binding: Reversibility

It can also be important to release bound ions from AuNPs. The influence of temperature on the ion-capturing mechanism was examined as a method to release ions. The initial conductivity of each hexanethioUEG AuNP solution was measured at room temperature, and then a stoichiometric amount of zinc, cadmium and cesium ions was added into each EG AuNP solutions, i.e. zinc ions into hexanethiol/EG1 AuNPs, cadmium ions to hexanethiol/EG2 AuNPs, and cesium ions to hexanethiol/EG3 AuNPs. Then, the solution was cooled to 0° C., and the conductivity was measured after recovering to room temperature within several minutes. Moreover, the solution was also heated to 80° C., and the conductivity was measured at room temperature in the same way. It was shown that the conductivity was increased when the temperature was raised to 80° C., without further addition of ion solution, as shown in FIG. 14A. This means that the bound ions were released from the AuNPs, induced by the increase of entropic contribution of ligands due to high temperature. This ion-releasing phenomenon by high temperature has also been observed in crown ether (Warshawsky, A.; Kahana, N. Journal of the American Chemical Society 1982, 104, 2663-2664, which is incorporated by reference in its entirety).

Furthermore, when the temperature was lowered to 0° C., the conductivity was reduced again, indicating that the released ions were bound again by AuNPs. This procedure was repeated three times, showing that the ion-capturing mechanism depends on temperature and it is also reversible. During the temperature-regulating procedure, the conductivity was measured after temperature was recovered to room temperature, controlled by a water-bath at room temperature, to minimize the temperature effect on the conductivity. To demonstrate that this conductivity change was caused by the ion capturing and releasing process, not just thermal effect, the same test has been done without AuNPs. In FIG. 14B, the conductivity slightly altered during the temperature-regulating procedure, but the amount of change was much smaller, compared to the result of the system with AuNPs.

Other embodiments are within the scope of the following claims.

What is claimed is:

1. An ion-binding nanoparticle comprising:
a nanoparticle core;
a first plurality of ligands on the core, wherein the first plurality of ligands includes a moiety capable of interacting with an ion; and
a second plurality of ligands different from the first plurality of the ligands on the core;
wherein the first plurality of ligands and the second plurality of ligands are arranged in a pattern on the core; and
wherein the first plurality of ligands has formula (I):
Z-L-[X—(CR_{d}Rₑ)ᵢ]ⱼ—R₁ (I)
wherein
Z is —SH, —OH, —NRₐH, —COOH, —P(O)₂OH, —S(O)OH, —S(O)₂OH, —NC, or —CN;
L is a C₁ to C₁₂ alkylene, cycloalkylene, alkenylene, alkynylene, or arylene group, wherein in L is optionally substituted by 0 to 10 groups selected from R_{b};
Each X, independently, is —O—, —S—, or —NRa-;
R₁ is —H, halo, cyano, nitro, —OR_{c}, —SR_{c}, or —NRₐR_{c};
or R₁ is a C₁ to C₆ alkyl, cycloalkyl, alkenyl, alkynyl, aryl, heteroaryl, or heterocyclyl group, wherein each of alkyl, cycloalkyl, alkenyl, alkynyl, aryl, heteroaryl, and heterocyclyl are optionally substituted by 0 to 3 groups selected from R_{b};
each Rₐ, independently, is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{b}, independently, is —H, halo, cyano, nitro, —OR_{f}, —SR_{f}, —NR_{f}R_{g}, oxo, alkyl, cycloalkyl, alkenyl, alkynyl, aryl, heteroaryl, or heterocyclyl;
R_{c} is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{d}, independently, is —H, halo, or C₁ to C₄ alkyl, wherein alkyl is optionally substituted by halo, —OR_{c}, —SR_{c}, or —NRₐR_{c};
each Rₑ, independently, is —H, halo, or C₁ to C₄ alkyl, wherein alkyl is optionally substituted by halo, —OR_{c}, —SR_{c}, or —NRₐR_{c};
each R_{f}, independently, is —H, alkyl, cycloalkyl, alkenyl, alkynyl, aryl, heteroaryl, or heterocyclyl;
each R_{g}, independently, is —H, alkyl, cycloalkyl, alkenyl, alkynyl, aryl, heteroaryl, or heterocyclyl;
each i, independently, is 0, 1, 2, 3, or 4; and
j is 0, 1, 2, 3, 4, 5, or 6.

2. The nanoparticle of claim 1, wherein the first plurality of ligands are substantially more hydrophilic than the second plurality of ligands.

3. The nanoparticle of claim 2, wherein the second plurality of ligands has formula (II):
Z-L-[X—(CR_{d}Rₑ)ᵢ]ⱼ—R₁ (II)
wherein
Z is —SH, —OH, —NRₐH, —COOH, —P(O)₂OH, —S(O)OH, —S(O)₂OH, —NC, or —CN;
L is a C₁ to C₁₂ alkylene, cycloalkylene, alkenylene, alkynylene, or arylene group, wherein in L is optionally substituted by 0 to 10 groups selected from R_{b};
Each X, independently, is —O—, —S—, or —NRa-;
R₁ is —H, halo, cyano, nitro, —OR_{c}, —SR_{c}, or —NRₐR_{c};
or R₁ is a C₁ to C₆ alkyl, cycloalkyl, alkenyl, alkynyl, aryl, heteroaryl, or heterocyclyl group, wherein each of alkyl, cycloalkyl, alkenyl, alkynyl, aryl, heteroaryl, and heterocyclyl are optionally substituted by 0 to 3 groups selected from R_{b};
each Rₐ, independently, is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{b}, independently, is —H, halo, cyano, nitro, —OR_{f}, —SR_{f}, —NR_{f}R_{g}, oxo, alkyl, cycloalkyl, alkenyl, alkynyl, aryl, heteroaryl, or heterocyclyl;
R_{c} is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{d}, independently, is —H, halo, or C₁ to C₄ alkyl, wherein alkyl is optionally substituted by halo, —OR_{c}, —SR_{c}, or —NRₐR_{c};
each Rₑ, independently, is —H, halo, or C₁ to C₄ alkyl, wherein alkyl is optionally substituted by halo, —OR_{c}, —SR_{c}, or —NRₐR_{c};
each R_{f}, independently, is —H, alkyl, cycloalkyl, alkenyl, alkynyl, aryl, heteroaryl, or heterocyclyl;
each R_{g}, independently, is —H, alkyl, cycloalkyl, alkenyl, alkynyl, aryl, heteroaryl, or heterocyclyl;
each i, independently, is 0, 1, 2, 3, or 4; and
j is 0, 1, 2, 3, 4, 5, or 6.

4. The nanoparticle of claim 3, wherein the moiety capable of interacting with an ion includes an ether oxygen, a hydroxyl group, or both.

5. The nanoparticle of claim 3, wherein the moiety capable of interacting with an ion includes from 2 to 6 ether, thioether, or amino functionalities.

6. The nanoparticle of claim 5, wherein the first plurality of ligands and the second plurality of ligands have the same values of Z and L.

7. The nanoparticle of claim 6, wherein, for the second plurality of ligands having formula (II), j is zero and R¹ is not H, halo, unsubstituted alkyl or unsubstituted aryl.

8. The nanoparticle of claim 7, wherein the second plurality of ligands is free of a moiety capable of interacting with an ion.

9. The nanoparticle of claim 8, wherein the nanoparticle core is a gold nanoparticle.

10. The nanoparticle of claim 9, wherein Z is —SH.

11. A method of making an ion-binding nanoparticle comprising:
contacting a nanoparticle core with a first plurality of ligands, wherein the first plurality of ligands includes a moiety capable of interacting with an ion;
contacting the nanoparticle core with a second plurality of ligands different from the first plurality of the ligands; and
wherein the first plurality of ligands and the second plurality of ligands are selected so as to form a pattern on the core; and
wherein the first plurality of ligands has formula (I):
Z-L-[X—(CR_{d}Rₑ)ᵢ]ⱼ—R₁ (I)
wherein
Z is —SH, —OH, —NRₐH, —COOH, —P(O)₂OH, —S(O)OH, —S(O)₂OH, —NC, or —CN;
L is a C₁ to C₁₂ alkylene, cycloalkylene, alkenylene, alkynylene, or arylene group, wherein in L is optionally substituted by 0 to 10 groups selected from R_{b};
Each X, independently, is —O—, —S—, or —NRa-;
R₁ is —H, halo, cyano, nitro, —OR_{c}, —SR_{c}, or —NRₐR_{c};
or R₁ is a C₁ to C₆ alkyl, cycloalkyl, alkenyl, alkynyl, aryl, heteroaryl, or heterocyclyl group, wherein each of alkyl, cycloalkyl, alkenyl, alkynyl, aryl, heteroaryl, and heterocyclyl are optionally substituted by 0 to 3 groups selected from R_{b};
each Rₐ, independently, is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{b}, independently, is —H, halo, cyano, nitro, —OR_{f}, —SR_{f}, —NR_{f}R_{g}, oxo, alkyl, cycloalkyl, alkenyl, alkynyl, aryl, heteroaryl, or heterocyclyl;
R_{c} is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{d}, independently, is —H, halo, or C₁ to C₄ alkyl, wherein alkyl is optionally substituted by halo, —OR_{c}, —SR_{c}, or —NRₐR_{c};
each Rₑ, independently, is —H, halo, or C₁ to C₄ alkyl, wherein alkyl is optionally substituted by halo, —OR_{c}, —SR_{c}, or —NRₐR_{c};
each R_{f}, independently, is —H, alkyl, cycloalkyl, alkenyl, alkynyl, aryl, heteroaryl, or heterocyclyl;
each R_{g}, independently, is —H, alkyl, cycloalkyl, alkenyl, alkynyl, aryl, heteroaryl, or heterocyclyl;
each i, independently, is 0, 1, 2, 3, or 4; and
j is 0, 1, 2, 3, 4, 5, or 6.

12. The method of claim 11, wherein the first plurality of ligands is hydrophilic and the second plurality of ligands is hydrophobic.

13. The method of claim 11, wherein the first plurality of ligands is longer than the second plurality of ligands.

14. The method of claim 11, wherein the nanoparticle core is a gold nanoparticle.

15. The method of claim 14, wherein the second plurality of ligands has formula (II):
Z-L-[X—(CR_{d}Rₑ)ᵢ]ⱼ—R₁ (II)
wherein
Z is —SH, —OH, —NRₐH, —COOH, —P(O)₂OH, —S(O)OH, —S(O)₂OH, —NC, or —CN;
L is a C₁ to C₁₂ alkylene, cycloalkylene, alkenylene, alkynylene, or arylene group, wherein in L is optionally substituted by 0 to 10 groups selected from R_{b};
Each X, independently, is —O—, —S—, or —NRa-;
R₁ is —H, halo, cyano, nitro, —OR_{c}, —SR_{c}, or —NRₐR_{c};
or R₁ is a C₁ to C₆ alkyl, cycloalkyl, alkenyl, alkynyl, aryl, heteroaryl, or heterocyclyl group, wherein each of alkyl, cycloalkyl, alkenyl, alkynyl, aryl, heteroaryl, and heterocyclyl are optionally substituted by 0 to 3 groups selected from R_{b};
each Rₐ, independently, is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{b}, independently, is —H, halo, cyano, nitro, —OR_{f}, —SR_{f}, —NR_{f}R_{g}, oxo, alkyl, cycloalkyl, alkenyl, alkynyl, aryl, heteroaryl, or heterocyclyl;
R_{c} is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{d}, independently, is —H, halo, or C₁ to C₄ alkyl, wherein alkyl is optionally substituted by halo, —OR_{c}, —SR_{c}, or —NRₐR_{c};
each Rₑ, independently, is —H, halo, or C₁ to C₄ alkyl, wherein alkyl is optionally substituted by halo, —OR_{c}, —SR_{c}, or —NRₐR_{c};
each R_{f}, independently, is —H, alkyl, cycloalkyl, alkenyl, alkynyl, aryl, heteroaryl, or heterocyclyl;
each R_{g}, independently, is —H, alkyl, cycloalkyl, alkenyl, alkynyl, aryl, heteroaryl, or heterocyclyl;
each i, independently, is 0, 1, 2, 3, or 4; and
j is 0, 1, 2, 3, 4, 5, or 6.

16. The method of claim 15, wherein the first plurality of ligands and the second plurality of ligands have the same values of Z and L.

17. A method of selectively binding ions comprising contacting an ion-containing composition with an ion-binding nanoparticle including:
a nanoparticle core;
a first plurality of ligands on the core, wherein the first plurality of ligands includes a moiety capable of interacting with an ion; and
a second plurality of ligands different from the first plurality of the ligands on the core;
wherein the first plurality of ligands and the second plurality of ligands are arranged in a pattern on the core; and
wherein the first plurality of ligands has formula (I):
Z-L-[X—(CR_{d}Rₑ)ᵢ]ⱼ—R₁ (I)
wherein
Z is —SH, —OH, —NRₐH, —COOH, —P(O)₂OH, —S(O)OH, —S(O)₂OH, —NC, or —CN;
L is a C₁ to C₁₂ alkylene, cycloalkylene, alkenylene, alkynylene, or arylene group, wherein in L is optionally substituted by 0 to 10 groups selected from R_{b};
Each X, independently, is —O—, —S—, or —NRa-;
R₁ is —H, halo, cyano, nitro, —OR_{c}, —SR_{c}, or —NRₐR_{c};
or R₁ is a C₁ to C₆ alkyl, cycloalkyl, alkenyl, alkynyl, aryl, heteroaryl, or heterocyclyl group, wherein each of alkyl, cycloalkyl, alkenyl, alkynyl, aryl, heteroaryl, and heterocyclyl are optionally substituted by 0 to 3 groups selected from R_{b};
each Rₐ, independently, is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{b}, independently, is —H, halo, cyano, nitro, —OR_{f}, —SR_{f}, —NR_{f}R_{g}, oxo, alkyl, cycloalkyl, alkenyl, alkynyl, aryl, heteroaryl, or heterocyclyl;
Rₑ is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{d}, independently, is —H, halo, or C₁ to C₄ alkyl, wherein alkyl is optionally substituted by halo, —OR_{c}, —SR_{c}, or —NRₐR_{c};
each Rₑ, independently, is —H, halo, or C₁ to C₄ alkyl, wherein alkyl is optionally substituted by halo, —OR_{c}, —SR_{c}, or —NRₐR_{c};
each R_{f}, independently, is —H, alkyl, cycloalkyl, alkenyl, alkynyl, aryl, heteroaryl, or heterocyclyl;
each R_{g}, independently, is —H, alkyl, cycloalkyl, alkenyl, alkynyl, aryl, heteroaryl, or heterocyclyl;
each i, independently, is 0, 1, 2, 3, or 4; and
j is 0, 1, 2, 3, 4, 5, or 6.

18. The method of claim 17, wherein the first plurality of ligands and the second plurality of ligands are selected such that the nanoparticle has a predetermined affinity for a predetermined ion.

19. The method of claim 18, wherein the predetermined ion is Li⁺, Na⁺, K⁺, Rb⁺, Cs⁺, Ca²⁺, Sr²⁺, Cu²⁺, Fe³⁺, Zn²⁺, Cr³⁺, Cd²⁺, or CH₃Hg⁺, or a combination thereof.

20. The method of claim 17, further comprising releasing the ions from the nanoparticle after binding.

21. The method of claim 20, wherein releasing the ions includes heating the nanoparticle.

22. The method of claim 21, wherein the second plurality of ligands has formula (II):
Z-L-[X—(CR_{d}Rₑ)ᵢ]ⱼ—R₁ (II)
wherein
Z is —SH, —OH, —NRₐH, —COOH, —P(O)₂OH, —S(O)OH, —S(O)₂OH, —NC, or —CN;
L is a C₁ to C₁₂ alkylene, cycloalkylene, alkenylene, alkynylene, or arylene group, wherein in L is optionally substituted by 0 to 10 groups selected from R_{b};
each X, independently, is —O—, —S—, or —NRa-;
R₁ is —H, halo, cyano, nitro, —ORₑ, —SR_{c}, or —NRₐR_{c};
or R₁ is a C₁ to C₆ alkyl, cycloalkyl, alkenyl, alkynyl, aryl, heteroaryl, or heterocyclyl group, wherein each of alkyl, cycloalkyl, alkenyl, alkynyl, aryl, heteroaryl, and heterocyclyl are optionally substituted by 0 to 3 groups selected from R_{b};
each Rₐ, independently, is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{b}, independently, is —H, halo, cyano, nitro, —OR_{f}, —SR_{f}, —NR_{f}R_{g}, oxo, alkyl, cycloalkyl, alkenyl, alkynyl, aryl, heteroaryl, or heterocyclyl;
R_{c} is —H, alkyl, cycloalkyl, alkenyl, alkynyl, aryl, heteroaryl, or heterocyclyl, wherein each of alkyl, cycloalkyl, alkenyl, alkynyl, aryl, heteroaryl, and heterocyclyl are optionally substituted by 0 to 3 groups selected from R_{b};
each R_{d}, independently, is —H, halo, or C₁ to C₄ alkyl, wherein alkyl is optionally substituted by halo, —OR_{c}, —SR_{c}, or —NRₐR_{c};
each Rₑ, independently, is —H, halo, or C₁ to C₄ alkyl, wherein alkyl is optionally substituted by halo, —OR_{c}, —SR_{c}, or —NRₐR_{c};
each R_{f}, independently, is —H, alkyl, cycloalkyl, alkenyl, alkynyl, aryl, heteroaryl, or heterocyclyl;
each R_{g}, independently, is —H, alkyl, cycloalkyl, alkenyl, alkynyl, aryl, heteroaryl, or heterocyclyl;
each i, independently, is 0, 1, 2, 3, or 4; and
j is 0, 1, 2, 3, 4, 5, or 6.

23. The method of claim 21, wherein the first plurality of ligands and the second plurality of ligands have the same values of Z and L.

24. The method of claim 17, further comprising binding to the nanoparticle a species associated with the selectively bound ion.

25. The method of claim 24, wherein the species associated with the selectively bound ion is a counterion.

26. The method of claim 18, further comprising selectively removing the selectively bound predetermined ions from the ion-containing composition.

27. The method of claim 18, further comprising selectively determining the presence, absence, or concentration of the predetermined ions in the ion-containing composition.
